(19) United States
(12) Patent Application Publication
(10) Pub. No.: US 2016/0039866 A1
(43) Pub. Date: Feb. 11, 2016

(54) 16- AND 17- DEUTERATED ESTROGEN-3-SULFAMATES AS ESTROGENIC AGENTS

(71) Applicant: UNIVERSITY OF BATH, Bath, Somerset (GB)

(72) Inventor: Barry Victor Lloyd POTTER, Bath (GB)

(21) Appl. No.: 14/767,175

(22) PCT Filed: Feb. 10, 2014
(86) PCT No.: PCT/GB2014/050378

§ 371 Date: Aug. 11, 2015

(30) Foreign Application Priority Data
Feb. 11, 2013 (GB) 1302368.4

(51) Int. Cl. C07J 41/00

(52) U.S. Cl. CPC C07J 41/0072

(57) ABSTRACT

The present invention relates to novel derivatives of estradiol, in particular to deuterated derivatives of estradiol sulfamates. The present invention also relates to compositions comprising said novel derivatives, as well as to uses of said novel derivatives and compositions comprising said derivatives.

### FIELD OF THE INVENTION

The present invention relates to novel derivatives of estradiol, in particular to deuterated derivatives of estradiol. The present invention also relates to compositions comprising said novel derivatives, as well as to uses of said novel derivatives and compositions comprising said derivatives.

### BACKGROUND TO THE INVENTION

Estrogens play a major role in hormonal contraception, in menopausal hormone replacement therapy (HRT), and for treating gynaecological diseases (e.g. mammary carcinoma) and andrological diseases (e.g. prostatic carcinoma). For HRT and contraception, estrogens are mainly used together with a gestagen, e.g. levonorgestrel, desogestrel, norethisterone, cyproterone acetate, chlormadinone acetate, dienogest.

When used for contraception, estrogens are needed for safely suppressing follicle maturation and ovulation, but in addition they replace the endogenous ovarian secretion of estradiol which is suppressed to a major extent. This replacement is important for maintaining an artificial menstrual cycle and other genital functions, which could not be done to any satisfactory extent by just using a gestagen.

In addition, endogenous and exogenous estrogens fulfil important central nervous and metabolic functions in the female organism: normal estrogen levels make a decisive contribution to a woman's well-being. Their presence in the system counteracts the development of cardiovascular diseases through various mechanisms: generation of “favourable” lipoprotein patterns in the blood, inhibition of lipid deposits in the walls of blood vessels, reduction in blood pressure by favourably influencing the vascular tonus, lowering of the perfusion resistance in essential vascular sectors, attenuation of contractile stimuli at the vascular muscle. The tunicae intimatae, when influenced by estrogens, release factors that counteract the formation of thrombi. Estrogens are also indispensable for preserving the bone structure in women. Their absence may result in destruction of the bone (osteoporosis). These latter “central nervous” and “metabolic” effects of estrogens are a main aspect of HRT. It can be considered that estrogens have analogous functions in the male organism, and that their withdrawal results in similar disorders as in women. One difference between the two sexes is that hormone production in males ceases less regularly and at a later age than that in women.

But notwithstanding all positive aspects of estrogen therapy there are unsolved problems, too, which restrict the therapeutic use of estrogens or entail undesired effects.

The known estrogens show pharmacokinetic deficits. When taken orally, natural estrogens (estradiol, oestrone, oestrone sulphate, esters of estradiol, oestriol, conjugated equine estrogens) are either bioavailable only to a low degree and/or exhibit marked inter-individual variability. Fast elimination of the substances from the blood is another problem. Plasma levels may vary so much from person to person that general dosage recommendations may be sub-optimal in many women. Estrogen replacement under HRT therefore often has to be adjusted to the individual.

The same is true of synthetic estrogens. The most widely used synthetically altered estrogenic steroid is ethinyl estradiol (EE). This estrogen is dominant in female oral hormonal contraception. Apart from EE, mestranol is used in a few cases; this is a methylated “prodrug” that is metabolised to EE in the organism. When applied orally to humans, EE has a much better bioavailability than the natural estrogens mentioned above, but its oral bioavailability also varies to a large extent from individual to individual. Several authors have pointed to this as well as to the fact that concentrations in the blood proved to be highly irregular after oral application of this substance (Goldzieher, J. W. 1989, Goldzieher, J. W. 1990, Humpel, M. 1987, Kuhnz, 1993).

In addition, the known estrogens show pharmacodynamic deficits. After resorption from the intestinal lumen, orally applied active ingredients enter the organism via the liver. This fact is of specific importance for estrogenic agents as the liver is a target organ for estrogens; oral intake of estrogens results in strong estrogenic effects in the liver. The secretion activity that is controlled by estrogens in the human liver includes synthesis of transfer proteins CBG, SHBG, TBG, angiotensinogen, several factors that are important for the physiology of blood clotting, and lipoproteins. If natural estrogens are introduced to the female organism while avoiding passage through the liver (e.g. by transdermal application), the liver functions mentioned remain virtually unchanged. Therapeutically equivalent doses of natural estrogens (see definition above), when applied orally, result in clear responses of hepatic parameters: increase of SHBG, CBG, angiotensinogen, HDL (high density lipoprotein). These hepatic effects of estrogen are clearly stronger when, instead of natural estrogens, equine estrogen formulations (so-called conjugated estrogens) are used (Campbell, S. et al., 1981). Ethinyl estradiol and diethylstilbestrol (DES) have an even greater hepatic oestrogenicity.

Synthetic estrogens, however, may also suffer from a number of additional drawbacks. When referring to antigonadotropic properties, EE is about 4 to 18 times more estrogenic in the liver than orally applied natural estrogens are (Campbell, S. et al., 1981). Furthermore, some synthetic estrogens are thought to be associated with an increased risk of developing venous thromboembolism (blood clots).

Accordingly, efforts have been made to prepare estrogens which overcome some of the above mentioned drawbacks. One class of compounds that have been postulated in this regard are estrogen sulfamates. Estrogen sulfamates represent a new class of estrogen that appear to transit the liver without undergoing first-pass inactivation, by binding to carbonic anhydrase II in red blood cells. The compound is then cleaved by the steroid sulfatase enzyme to release the active metabolite.

In view of the above drawbacks with regard to synthetic estrogens and the deficiencies with regard to the oral bioavailability of some natural estrogens, there is a desire to provide compounds which can be considered therapeutically equivalent to natural estrogens which do not suffer from the drawbacks of synthetic estrogens and yet which have an acceptable bioavailability and/or half-life in vivo.

Accordingly, the present invention aims to provide improved estrogen derivatives.

### SUMMARY ASPECTS OF THE PRESENT INVENTION

In a first aspect, the present invention relates to a compound of formula (I)

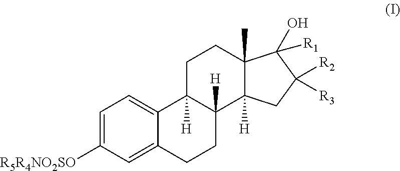

or a pharmaceutically acceptable salt thereof, wherein:
R₁, R₂ and R₃ are each independently selected from H and deuterium;
R₄ and R₅ are each independently selected from H, deuterium, optionally deuterated C₁-C₄ alkyl and optionally deuterated C₁-C₄ acyl; and
at least one of R₁, R₂ or R₃ is deuterium.

In a further aspect, the present invention relates to a composition comprising a compound of formula (I) as defined above, or a pharmaceutically acceptable salt thereof, and at least one pharmaceutically acceptable carrier.

In a further aspect, the present invention relates to a compound of formula (I) as defined above, or a pharmaceutically acceptable salt thereof, for use as a medicament.

In a further aspect, the present invention relates to a compound of formula (I) as defined above, or a pharmaceutically acceptable salt thereof, for use in hormone replacement therapy.

In a further aspect, the present invention relates to a compound of formula (I) as defined above, or a pharmaceutically acceptable salt thereof, for use as a contraceptive.

In a further aspect, the present invention relates to the use of a compound of formula (I) as defined above, or a pharmaceutically acceptable salt thereof, as a contraceptive.

### DETAILED DESCRIPTION OF THE INVENTION

In a first aspect, the present invention relates to a compound of formula (I)

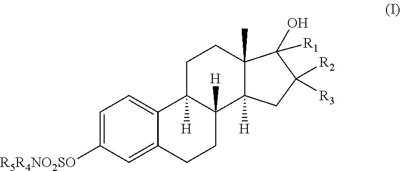

or a pharmaceutically acceptable salt thereof, wherein:
R₁, R₂ and R₃ are each independently selected from H and deuterium;
R₄ and R₅ are each independently selected from H, deuterium, optionally deuterated C₁-C₄ alkyl and optionally deuterated C₁-C₄ acyl; and
at least one of R₁, R₂ or R₃ is deuterium.

Thus, it will be appreciated that the compounds of formula (I) are sulfamates, i.e. the group —OSO₂NR₄R₅ is a sulfamate group.

It will also be appreciated that the compounds of formula (I) are deuterated compounds. In this regard, “deuterated” means that at least one of the atoms in the compound is deuterium in an abundance that is greater than the natural abundance of deuterium (typically 0.015%).

“Deuterium” (also referred to as “D” or “d”) refers to an isotope of hydrogen whose nucleus contains one proton and one neutron. When a particular position is designated as having deuterium, it is understood that the abundance of deuterium at that position is greater than the natural abundance of deuterium (typically 0.015%).

It is understood that deuterium in this context is regarded as a substituent of the compounds of the present invention. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted as deuterium, such compound has an isotopic enrichment factor for each designated deuterium atom of at least 1000 (15% deuterium incorporation at each designated deuterium atom), at least 1500 (22.5% deuterium incorporation at each designated deuterium atom), at least 2000 (30% deuterium incorporation at each designated deuterium atom), at least 2500 (37.5% deuterium incorporation at each designated deuterium atom), at least 3000 (45% deuterium incorporation at each designated deuterium atom), at least 3340 (50.1% deuterium incorporation at each designated deuterium atom), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “compound,” as used herein, refers to a collection of molecules having an identical chemical structure, except that there may be isotopic variation among the constituent atoms of the molecules. Thus, the extent of deuteration at each designated atom is not necessarily the same. For example, where a compound according to the present invention contains multiple deuterium atoms, an isotopic enrichment factor for one designated deuterium atom may be 4000 (60% deuterium incorporation) and an isotopic enrichment factor for another designated deuterium atom may be 4500 (67.5% deuterium incorporation). Thus, such a compound is considered to have an isotopic enrichment factor of at least 4000.

The structural formula depicted herein may or may not indicate whether atoms at certain positions are isotopically enriched. In a most general embodiment, when a structural formula is silent with respect to whether a particular position is isotopically enriched, it is to be understood that the stable isotopes at the particular position are present at natural abundance, or, alternatively, that that particular position is isotopically enriched with one or more naturally occurring stable isotopes. In a more specific embodiment, the stable isotopes are present at natural abundance at all positions in a compound not specifically designated as being isotopically enriched.

As mentioned above, the present invention relates to a compound of formula (I)

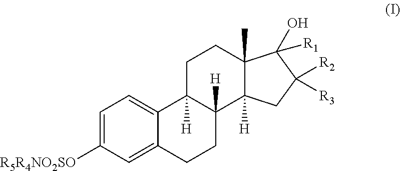

or a pharmaceutically acceptable salt thereof, wherein:
R₁, R₂ and R₃ are each independently selected from H and deuterium;
R₄ and R₅ are each independently selected from H, deuterium, optionally deuterated C₁-C₄ alkyl and optionally deuterated C₁-C₄ acyl; and
at least one of R₁, R₂ or R₃ is deuterium.

In one embodiment, R₂ is deuterium and R₁ and R₃ are each H. In one embodiment, R₃ is deuterium and R₁ and R₂ are each H. In one preferred embodiment, R₁ is deuterium and R₂ and R₃ are each H.

In one embodiment, at least two of R₁, R₂ or R₃ are deuterium.

In one embodiment, R₂ is H and R₁ and R₃ are each deuterium. In one embodiment, R₃ is H and R₁ and R₂ are each deuterium. In these embodiments, the deuterium at the C-16 carbon may be in either the 16-alpha or 16-beta stereoconfiguration.

In one preferred embodiment, R₁ is H and R₂ and R₃ are each deuterium.

In one preferred embodiment, each of R₁, R₂ and R₃ are simultaneously deuterium.

As described above, R₄ and R₅ are each independently selected from H, deuterium, optionally deuterated C₁-C₄ alkyl and optionally deuterated C₁-C₄ acyl.

In one embodiment, R₄ and R₅ are each simultaneously H. In one embodiment, R₄ and R₅ are each simultaneously deuterium. In one embodiment, one of R₄ and R₅ is deuterium and the other is H.

In one embodiment, R₄ and R₅ are each simultaneously C₁-C₄ alkyl, preferably methyl, ethyl or propyl. In one embodiment, R₄ and R₅ are independently C₁-C₄ alkyl, preferably methyl, ethyl or propyl.

As mentioned above, R₄ and R₅ may be selected from deuterated C₁-C₄ alkyl. In this regard, deuterated C₁-C₄ alkyl means that the alkyl group contains from 1 to 9 deuterium atoms.

In one embodiment, R₄ and R₅ are independently deuterated C₁-C₄ alkyl, preferably deuterated methyl, ethyl or propyl. In one embodiment, R₄ and R₅ are each simultaneously deuterated C₁-C₄ alkyl, preferably deuterated methyl, ethyl or propyl.

In one embodiment, R₄ and R₅ are each simultaneously C₁-C₄ acyl. In one embodiment, R₄ and R₅ are independently C₁-C₄ acyl. In this regard, deuterated C₁-C₄ acyl means that the carbon chain of the acyl group has from 1 to 4 carbon atoms which may contain from 1 to 9 deuterium atoms.

In one embodiment, the present invention relates to a compound of formula (II)

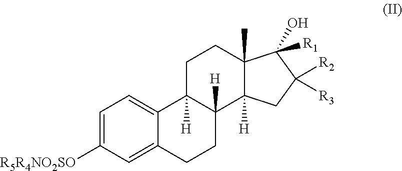

or a pharmaceutically acceptable salt thereof, wherein R₁ to R₅ are as defined above.

In a preferred embodiment, the present invention relates to a compound of formula (III)

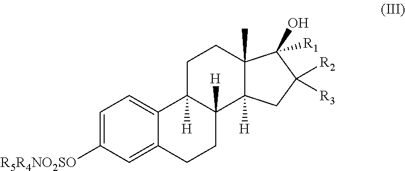

or a pharmaceutically acceptable salt thereof, wherein R₁ to R₅ are as defined above.

In one embodiment, the compound of the present invention is selected from:

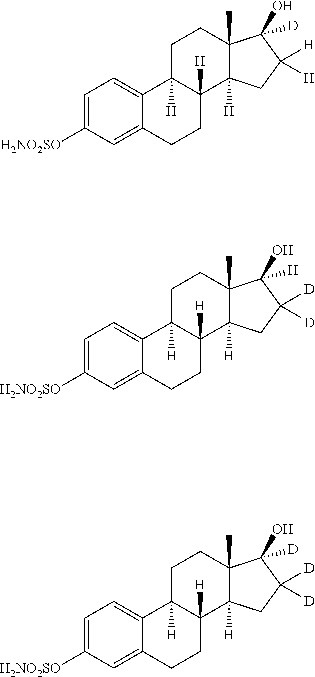

In any of the above embodiments, any atom not designated as deuterium can be considered to be present at its natural abundance.

Alternatively, in one embodiment, any hydrogen atom may optionally be substituted for a deuterium atom at an abundance that is greater than the natural abundance of deuterium (typically 0.015%).

### Some Advantages

Key advantages of the present invention include the improved pharmacokinetics and pharmacodynamics of the compound of the present invention.

One surprising and key advantage of the present invention is that the specific deuterated compounds of the present invention have a lower metabolism compared to non-deuterated estradiol sulfamate compounds.

A further key advantage of the present invention is that the sulfamate compounds exhibit low variation in concentration in plasma between individuals. The reduction in variation between individuals of estrogen levels in the blood particularly allows for the provision of a monoproduct for both hysterectomised and non-hysterectomised subjects without the need to develop separate products.

A further key advantage of the present invention is that the sulfamate compounds exhibit slow elimination from the subject. The composition of the present invention provides steady plasma levels of deuterated estradiol (E2).

Thus in a further aspect the present invention provides use of a compound as defined herein, preferably a composition as defined herein, in the manufacture of a medicament for bone protecting hormone replacement therapy without endometrial stimulation.

Another advantage is that a composition may be formulated without the incorporation of progestins.

Another advantage is that a composition may be formulated without the incorporation of gestagens.

### Hormone Replacement Therapy

The compound of the present invention may be formulated to provide a hormone replacement therapy. The compound will be formulated to contain the deuterated compound (the compound of formula I) in an amount such that, depending on the prescribed frequency of administration, the required daily dosage of deuterated compound is provided.

In one aspect the composition will be formulated to allow for daily administration. This composition may be formulated in combination with progestins.

In all aspects of the present invention, including daily oral or weekly administration, hormone replacement therapy is achieved in a manner more convenient than application of a transdermal patch, one typical administration route for HRT.

### Contraception

The compound of the present invention may be formulated to provide an oral contraceptive.

The compound will be formulated to contain the deuterated compound (the compound of formula I) in an amount such that, depending on the prescribed frequency of administration, the required dosage of deuterated compound is provided.

In one aspect the composition will be formulated to allow for daily administration. In one aspect the composition will be formulated to allow for weekly administration.

### Therapy

The compounds of the present invention may be used as therapeutic agents—i.e. in therapy applications.

The term “therapy” includes curative effects, alleviation effects, and prophylactic effects.

The therapy may be on humans or animals, preferably females, more preferably female humans.

### Pharmaceutical Compositions

The present invention also provides a pharmaceutical composition, which comprises a compound according to the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, as an interuterine system, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner. In one preferred embodiment the pharmaceutical composition is administered/formulated to be administered orally.

### Combination Pharmaceutical

The compound of the present invention may be used in combination with one or more other active agents, such as one or more other pharmaceutically active agents.

The compounds according to the invention may be used alone or in combination with a progestin, for example levonorgestrel, desogestrel, norethisterone, cyproterone acetate, chlormadinone acetate, or dienogest. Preferably the compounds according to the invention are used in the absence of a progestin. Thus, in a preferred aspect the composition of the present invention is substantially free of progestins.

In addition, or in the alternative, the compound of the present invention may be used in combination with a biological response modifier.

The term biological response modifier (“BRM”) includes cytokines, immune modulators, growth factors, haematopoiesis regulating factors, colony stimulating factors, chemotactic, haemolytic and thrombolytic factors, cell surface receptors, ligands, leukocyte adhesion molecules, monoclonal antibodies, preventative and therapeutic vaccines, hormones, extracellular matrix components, fibronectin, etc. For some applications, preferably, the biological response modifier is a cytokine. Examples of cytokines include: interleukins (IL)—such as IL-1, IL-2, IL-3, IL-4, IL-5, IL-6, IL-7, IL-8, IL-9, IL-10, IL-11, IL-12, IL-19; Tumour Necrosis Factor (TNF)—such as TNF-α; Interferon alpha, beta and gamma; TGF-β. For some applications, preferably the cytokine is tumour necrosis factor (TNF). For some applications, the TNF may be any type of TNF—such as TNF-α, TNF-β, including derivatives or mixtures thereof. More preferably the cytokine is TNF-α. Teachings on TNF may be found in the art—such as WO-A-98/08870 and WO-A-98/13348.

### Administration

The compositions of the present invention may be administered by direct injection. The composition may be formulated for parenteral, mucosal, intramuscular, intravenous, subcutaneous, intraocular or transdermal administration.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Aside from the typical modes of delivery—indicated above—the term “administered” also includes delivery by techniques such as lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestible solution; a parenteral route where delivery is by an injectable form, such as, for example, an intravenous, intramuscular or subcutaneous route.

Thus, for pharmaceutical administration, the compounds of the present invention can be formulated in any suitable manner utilising conventional pharmaceutical formulating techniques and pharmaceutical carriers, adjuvants, excipients, diluents etc. and usually for parenteral administration. Dosages may be given in single dose regimes, split dose regimes and/or in multiple dose regimes lasting over several days. For oral administration they may be formulated in tablets, capsules, solution or suspension. Alternatively the compounds will be formulated for parenteral administration in a suitable parenterally administrable carrier and providing single daily dosage rates. Effective daily doses will, however, vary depending on inherent activity of the active ingredient and on the bodyweight of the patient, such variations being within the skill and judgement of the physician.

The present invention will now be described with reference to the following non-limiting examples.

### EXAMPLES

### Experimental

### General Methods for Synthesis

All chemicals were purchased from either Aldrich Chemical Co. (Gillingham, UK) or Alfa Aesar (Heysham, UK). All organic solvents of AR grade were supplied by Fisher Scientific (Loughborough, UK). Melting points were determined using a Stanford Research Systems Optimelt MPA100 and are uncorrected. Thin layer chromatography (TLC) was performed on pre-coated aluminium plates (Merck, silica gel 60 F₂₅₄). Products were visualized either by UV irradiation at 254 nm and by staining with 5% w/v molybdophosphoric acid in ethanol, followed by heating. Flash column chromatography was performed on pre-packed columns (RediSep Rf) and gradient elution (solvents indicated in text) on the Combiflash RF system (Teledyne Isco). ¹H NMR spectra were recorded with a Bruker 400 or 500 MHz spectrometer. Chemical shifts are reported in parts per million (ppm, δ) relative to tetramethylsilane (TMS) as an internal standard. High resolution mass spectra were recorded on a Bruker MicroTOF with ESI.

### Synthesis of 17α-deuterium-estra-1,3,5(10)-triene-3,17-diol-3-O-sulfamate (3)

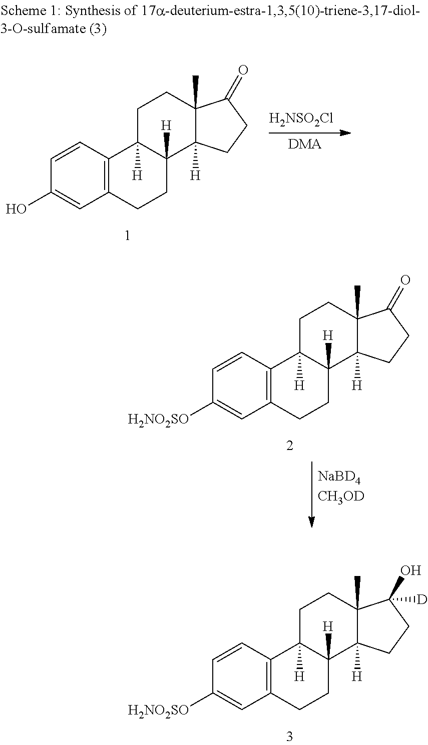

### Estra-1,3,5(10)-triene-17-one-3-O-sulfamate (2)

A solution of sulfamoyl chloride in toluene (6 mL, 0.63 M, 4.2 mmol) was concentrated in vacuo under 25° C. The residue was dissolved in anhydrous DMA (10 mL) at 0° C. and the mixture stirred for 15 minutes under an atmosphere of nitrogen. Estrone (1, 0.54 g, 2.0 mmol) was added and the mixture was stirred at room temperature for 24 hours, and then partitioned between EtOAc (50 mL) and water (50 mL). The aqueous layers were extracted with EtOAc (2×50 mL). The combined organic phase was washed with brine (100 mL), dried (MgSO₄), filtered and concentrated in vacuo. The product was purified using flash chromatography eluting with EtOAc/Petrol ether gradient to give white solid (0.57 g, 81%); mp 194-196° C. ¹H NMR (400 MHz, CDCl₃/DMSO-d₆): δ 0.79 (3H, s, CH₃), 1.22-1.63 (6H, m), 1.76-2.46 (7H, m), 2.77-2.85 (2H, m), 6.67 (2H, s, NH₂), 6.95 (1H, d, J=2.2 Hz), 6.97 (1H, dd, J=8.2, 2.3 Hz), 7.16 (1H, d, J=8.2 Hz). LC/MS (ESI) tᵣ=3.9 min; m/z 350.1 (M+H)⁺ , 100%).

### 17α-Deuterium-estra-1,3,5(10)-triene-3,17-diol-3-O-sulfamate (3)

A solution of estra-1,3,5(10)-triene-17-one-3-O-sulfamate (2, 410 mg, 1.17 mmol) in CH₃OD-D₂O (3:1, 20 mL) was stirred at room temperature under nitrogen for 30 minutes, concentrated in vacuo to dryness. The residue was dissolved in CH₃OD (20 mL), and NaBD₄ (94 mg, 2.2 mmol) was added. The mixture was refluxed under nitrogen for 1 hour, cooled and concentrated in vacuo to half of the volume, and diluted with water (15 mL). The white precipitate was collected and washed with water, dried in vacuo. The product was purified using flash chromatography eluting with EtOAc/DCM gradient to give white solid (285 mg, 69%); mp 198-200° C. ¹H NMR (400 MHz, DMSO-d₆): δ 0.72 (3H, s, CH₃), 1.12-1.50 (7H, m), 1.60-1.70 (1H, m), 1.80-2.00 (3H, m), 2.25 (1H, m), 2.36 (1H, m), 2.89 (2H, m), 4.56 (1H, s, OH), 7.00 (1H, d, J=2.5 Hz), 7.05 (1H, dd, J=8.3, 2.5 Hz), 7.40 (1H, d, J=8.6 Hz), 7.96 (2H, s, NH₂); HRMS (ESI) calcd. for C₁₈H₂₄DNNaO₄S (M+Na)⁺ 375.1465. found 375.1448.

### Synthesis of 16,16-Di-deuterium-estra-1,3,5(10)-triene-3,17-diol-3-O-sulfamate (6) and 16,16,17α-tri-deuterium-estra-1,3,5(10)-triene-3,17-diol-3-O-sulfamate (7)

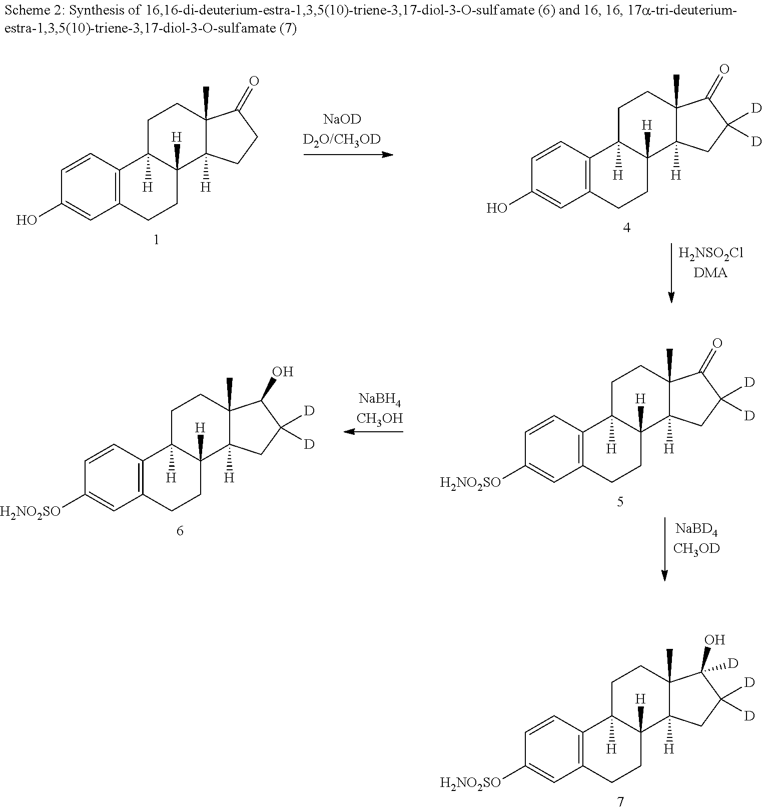

### 16,16-Di-deuterium-estra-1,3,5(10)-triene-17-one (4)

To a solution of estra-1,3,5(10)-triene-17-one (1, 1.0 g, 3.7 mmol) in CH₃OD (30 mL) was added NaOD in D₂O (1.52 g, 40%, 14.8 mmol). The mixture was refluxed under nitrogen for 24 hours, cooled and neutralized slowly with 10% H₂SO₄ to pH 7. The white precipitate was collected and washed with water, crystallised from ethanol (50 mL) and dried in vacuo to give white solid (1.0 g, 99%). mp 250-253° C. ¹H NMR (400 MHz, CDCl₃): δ 0.89 (3H, s, CH₃), 1.38-1.70 (6H, m), 1.99 (3H, m), 2.25-2.45 (2H, m), 2.89 (2H, m), 4.66 (1H, s, OH), 6.58 (1H, br s), 6.62 (1H, br d, J=8.5 Hz), 7.15 (1H, d, J=8.4 Hz); HRMS (ESI) calcd. for C₁₈H₂₀D₂NaO₂ (M+Na)⁺ 295.1643. found 295.1643.

### 16,16-Di-deuterium-estra-1,3,5(10)-triene-17-one-3-O-sulfamate (5)

A solution of sulfamoyl chloride in toluene (3 mL, 0.63 M, 2.1 mmol) was concentrated in vacuo under 25° C. The residue was dissolved in anhydrous DMA (5 mL) at 0° C. and the mixture stirred for 15 minutes under an atmosphere of nitrogen. 16,16-Di-deuterium-estra-1,3,5(10)-triene-17-one (4, 272 mg, 1.0 mmol) was added and the mixture was stirred at room temperature overnight, and then partitioned between EtOAc (30 mL) and water (30 mL). The aqueous layers were extracted with EtOAc (2×30 mL). The combined organic phase was washed with brine (60 mL), dried (MgSO₄), filtered and concentrated in vacuo. The product was purified using flash chromatography eluting with EtOAc/Petrol ether gradient to give white solid (200 mg, 57%); mp 192-194° C. ¹H NMR (400 MHz, CDCl₃/DMSO-d₆): δ 0.82 (3H, s, CH₃), 1.25-1.66 (6H, m), 1.96-1.99 (3H, m), 2.55-2.65 (2H, m), 2.78 (2H, m), 6.59 (2H, s, NH₂), 6.91 (1H, d, J=2.1 Hz), 6.93 (1H, dd, J=8.3, 2.2 Hz), 7.12 (1H, d, J=8.1 Hz); HRMS (ESI) calcd. for C₁₈H₂₁D₂NNaO₄S (M+Na)⁺ 374.1371. found 374.1375.

### 16,16-Di-deuterium-estra-1,3,5(10)-triene-3,17-diol-3-O-sulfamate (6)

To a solution of 16,16-di-deuterium-estra-1,3,5(10)-triene-17-one-3-O-sulfamate (5, 100 mg, 0.28 mmol) in CH₃OH (6 mL) was added NaBH₄ (38 mg, 1.0 mmol). The mixture was refluxed under nitrogen for 1 hour, cooled and acetic acid (0.3 mL) was added. The mixture was partitioned between EtOAc and water. The aqueous layers were extracted with EtOAc (2×30 mL). The combined organic phase was washed with brine (60 mL), dried (MgSO₄), filtered and concentrated in vacuo. The product was purified using flash chromatography eluting with EtOAc/DCM gradient to give white solid (69 mg, 70%); mp 197-200° C. ¹H NMR (500 MHz, DMSO-d₆): δ 0.72 (3H, s, CH₃), 1.15-1.50 (6H, m), 1.62 (1H, m), 1.85-2.00 (2H, m), 2.22 (1H, m), 2.49 (1H, m), 2.88 (2H, m), 3.55 (1H, m), 4.60 (1H, br s, OH), 6.95 (1H, br s), 7.05 (1H, br d, J=8.5 Hz), 7.35 (1H, d, J=8.5 Hz), 7.88 (2H, s, NH₂); HRMS (ESI) calcd, for C₁₈H₂₃D₂NNaO₄S (M+Na)⁺ 376.1528. found 376.1510.

### 16,16,17α-Tri-deuterium-estra-1,3,5(10)-triene-3,17-diol-3-O-sulfamate (7)

To a solution of 16,16-di-deuterium-estra-1,3,5(10)-triene-17-one-3-O-sulfamate (5, 150 mg, 0.43 mmol) in CH₃OD (15 mL) was added NaBD₄ (100 mg, 2.4 mmol). The mixture was refluxed under nitrogen for 1 hour, cooled and partitioned between EtOAc and water. The aqueous layers were extracted with EtOAc (2×30 mL). The combined organic phase was washed with brine (60 mL), dried (MgSO₄), filtered and concentrated in vacuo. The product was purified using flash chromatography eluting with EtOAc/DCM gradient to give white solid (110 mg, 72%); mp 195-197° C. ¹H NMR (400 MHz, DMSO-d₆): δ 0.72 (3H, s, CH₃), 1.10-1.45 (6H, m), 1.65 (1H, m), 1.90 (2H, m), 2.22 (1H, m), 2.38 (1H, m), 2.82 (2H, m), 4.49 (1H, s, OH), 6.96 (1H, br s), 7.00 (1H, br d, J=7.8 Hz), 7.33 (1H, d, J=7.8 Hz), 7.88 (2H, s, NH₂); HRMS (ESI) calcd. for C₁₈H₂₁D₃NO₄S (M−H)⁺ 353.1614. found 363.1608.

### Exemplary synthesis of 17α-deuterium-N,N-dialkyl-estra-1,3,5(10)-triene-3,17-diol-3-O-sulfamate (R₁ and R₂=alkyl)

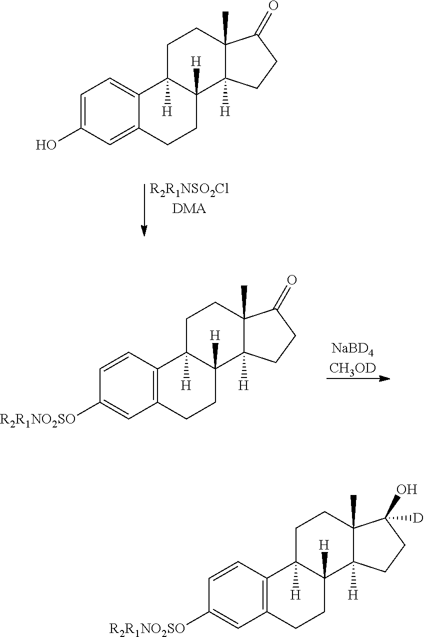

### Exemplary synthesis of 16,16-Di-deuterium-N,N-dialkyl-estra-1,3,6(10)-triene-3,17-diol-3-O-sulfamate and 16,16,17α-tri-deuterium-N,N-dialkyl-estra-1,3,5(10)-triene-3,17-diol-3-O-sulfamate (R₁ and R₂=alkyl)

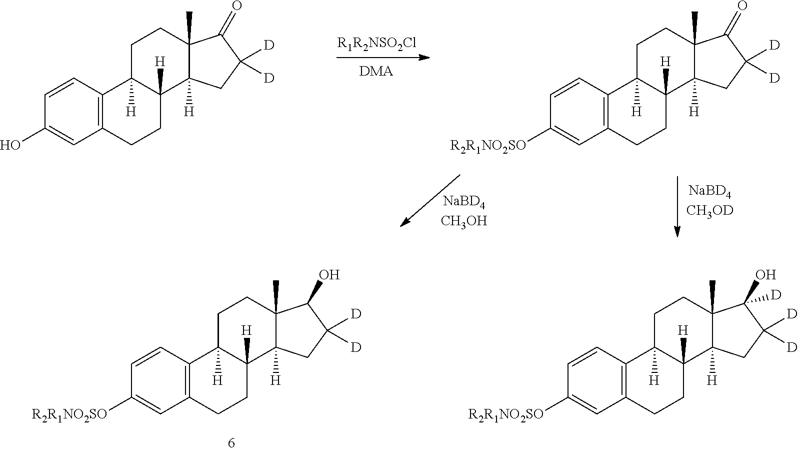

### Sulfamoylation

The sulfamoylation at the 3 position of deuterium labelled estrogens can also be performed using N-(tert-Butoxycarbonyl)-N-[(triethylenediammonium)sulfonyl]azanide according to the method reported in Org. Lett., 2012, 14 (10), pp 2626-2629.

### Deuteration

The skilled person will understand that the isotopic purity of deuterated compounds can be improved by the use of deuterated reagents used to make the compound and also by the efficiency of incorporation of deuterium in the various synthesis steps used to prepare the compound.

### Evaluation of Metabolic Stability

The resistance of these deuterated compounds to metabolism is examined using MCF-7 breast cancer cells transiently transfected with the human cDNA for the 17β-HSD2 enzyme. Separation of the resultant steroids by reverse phase HPLC over time allows calculation of the rate of metabolism.

### Transient Transfection and Assay Procedure:

MCF-7 cells are seeded into T-25 tissue culture flasks at 40% density. After 24 h, cells are transfected with 1 μg per flask of pAlter 17β-HSD2 combined with 3 μl Fugene and 100 μl FBS-free MEM. Compounds for study (10 μM) are added to cells or medium 48 h after transfection and cells and medium extracted with diethyl ether for HPLC analysis after incubation for 4 h.

### HPLC Analysis:

Estrogen sulfamates and their deuterated derivatives are analysed by reverse-phase HPLC using an Agilent 1100 Chemstation system. After evaporation of the diethyl ether the residues are reconstituted in mobile phase. Ammonium sulfate (0.02 M) is used as the aqueous phase and methanol or acetonitrile as the organic phase. Estrogen sulfamates are separated from their putative metabolites using a Gemini C18 column and the samples are analysed using a diode array detector. The areas under the resolved peaks are calculated to determine the percentage of compound metabolised by 17β-HSD2.

All publications and patents mentioned in the above specification are herein incorporated by reference.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A compound of formula (I) 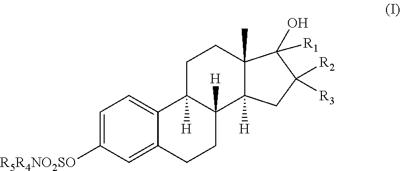
or a pharmaceutically acceptable salt thereof, wherein:
R₁, R₂, and R₃ are each independently H or deuterium;
R₄ and R₅ are each independently H, deuterium, optionally deuterated C₁-C₄ alkyl or optionally deuterated C₁-C₄ acyl;
and at least one of R₁, R₂, or R₃ is deuterium.

2. The compound of claim 1, wherein R₂ is deuterium and R₁ and R₃ are each H.

3. The compound of claim 1, wherein R₃ is deuterium and R₁ and R₂ are each H.

4. The compound of claim 1, wherein R₁ is deuterium and R₂ and R₃ are each H.

5. The compound of claim 1, wherein at least two of R₁, R₂ or R₃ are deuterium.

6. The compound of claim 5, wherein R₂ is H and R₁ and R₃ are each deuterium.

7. The compound of claim 5, wherein R₃ is H and R₁ and R₂ are each deuterium.

8. The compound of claim 5, wherein R₁ is H and R₂ and R₃ are each deuterium.

9. The compound of claim 1, wherein each of R₁, R₂ or R₃ are simultaneously deuterium.

10. The compound according to claim 1, wherein R₄ and R₅ are each simultaneously H.

11. The compound according to claim 1, wherein R₄ and R₅ are each simultaneously deuterium.

12. The compound according to claim 1, wherein said compound is:

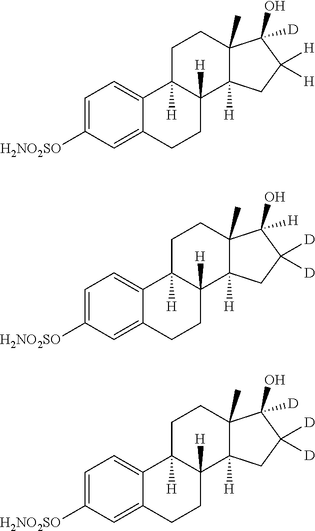

13. A The compound according to claim 1, wherein any atom not designated as deuterium is present at its natural abundance.

14. A composition comprising the compound of claim 1, or a pharmaceutically acceptable salt thereof, and at least one pharmaceutically acceptable carrier.

15. A medicament comprising the compound of claim 1, or a pharmaceutically acceptable salt thereof.

16. A method of hormone replacement therapy comprising administering to a patient in need thereof the compound of claim 1, or a pharmaceutically acceptable salt thereof.

17. A method of contraception comprising administering to a patient in need thereof the compound of claim 1, or a pharmaceutically acceptable salt thereof.

18. (canceled)

19. (canceled)
